(19) United States
(12) Patent Application Publication
(10) Pub. No.: US 2018/0228943 A1
(43) Pub. Date: Aug. 16, 2018

(54) ELECTRICALLY CONDUCTIVE COATING WITH GRADIENT OF PARTICLE CONTENT, IN PARTICULAR FOR MEDICAL DEVICES

(71) Applicant: Heraeus Deutschland GmbH & Co. KG, Hanau (DE)

(72) Inventors: Jens Trötzschel, Ronneburg (DE); Detlef Gaiser, Köln (DE); Stefan Schibli, Frankfurt (DE)

(21) Appl. No.: 15/897,246

(22) Filed: Feb. 15, 2018

(30) Foreign Application Priority Data
Feb. 16, 2017 (EP) 17156490.9

(51) Int. Cl. A61L 31/12; B33Y 30/00; B33Y 70/00; B33Y 10/00; B33Y 80/00; A61L 31/06; A61L 29/06; A61L 27/34; A61L 27/44; A61L 29/12; C09D 183/04

(52) U.S. Cl. CPC A61L 31/126; H01B 1/22; B33Y 70/00; B33Y 10/00; B33Y 80/00; A61L 31/06; A61L 29/06; A61L 27/34; A61L 27/443; A61L 29/126; C09D 183/04; A61L 2420/04; A61L 2420/06; A61L 2420/08; B33Y 30/00

(57) ABSTRACT

The invention relates to a composite (100), containing as mutually superimposed layers of a series of layers
a) a substrate (101), and
b) a first layer (102);
   wherein the first layer (102) contains
i) a first layer surface (103),
ii) a polymer, and
iii) a plurality of electrically conductive particles;
   wherein the first layer surface (103) is adjacent to the substrate (101); wherein at least in a first region, (104) the first layer (102) at a first distance (105) from the first layer surface (103) is characterized by a first content (403) of the electrically conductive particles; wherein at least in the first region (104), the first layer (102) at a further distance (106) from the first layer surface (103) is characterized by a further content of the electrically conductive particles; wherein the first content is less than the further content; and wherein the first distance (105) is less than the further distance (106). The invention further relates to an apparatus (600), a method (800), an electrical component (1200), an electrical device (1201), a 3D printer (1100) and a use.

The present invention relates to a composite, containing as mutually superimposed layers of a series of layers
a) a substrate, and
b) a first layer;
   wherein the first layer contains
i) a first layer surface,
ii) a polymer, and
iii) a plurality of electrically conductive particles;
   wherein the first layer surface is adjacent to the substrate; wherein at least in a first region, the first layer at a first distance from the first layer surface is characterized by a first content of the electrically conductive particles; wherein at least in the first region, the first layer at a further distance from the first layer surface is characterized by a further content of the electrically conductive particles; wherein the first content is less than the further content; wherein the first distance is less than the further distance. The invention further relates to an apparatus, a method, an electrical component, an electrical device, a 3D printer and a use.

In a variety of applications, electrically conductive coatings a few μm in thickness are used on a substrate. If such a thin electrically conductive coating is to be electrically contacted, this gives rise in the prior art to serious problems. If the electrically conductive coating is a metallic coating a few μm in thickness, it will tend to peel or form cracks when mechanically stressed. In this case, the mechanical stress can result from (elastic) deformation of the substrate or from mechanical contacting of the coating itself. The rather brittle metallic coating therefore cannot constitute a durable, easily electrically contactable coating of a substrate. Alternatively, electrically conductive plastics for the formation of coatings on substrates are known in the prior art. If these electrically conductive plastic coatings are mechanically contacted, for example via clamps or springs, there is considerable contact resistance to the contacting element, which in the case of layers that are only a few μm thick can exceed the electrical resistance of the layer itself. In the prior art, this contact resistance can only be reduced by application of contact lacquers. Both metallic and electrically conductive plastic coatings therefore show considerable drawbacks with respect to their electrical contacting.

In the prior art, this electrical contacting can be carried out by different methods. The welding or soldering known in this connection is not suitable for temperature-sensitive substrates because of the high temperatures involved. Moreover, it is virtually impossible to weld or solder coatings that are a few μm thick. Electrical contacting by means of electrically conductive epoxy resins is highly complex. Moreover, the coating in this case is irreversible, i.e. cannot be detached. In principle, mechanical contacts are detachable, but as mentioned above, they lead to considerable contact resistance in the case of electrically conductive plastic coatings. Finally, electrical contacting by bonding is known in the prior art. This coating method is generally suitable only for metallic coatings, wherein the coating must have a minimum surface area and must also be as flat as possible.

In general, an object of the present invention is to at least partially overcome a drawback of the prior art. A further object of the invention is to provide an electrically conductive coating superimposed on a substrate, wherein the coating provides the most advantageous combination possible of the following properties: high electrical conductivity, low contact resistance, high durability, high mechanical stability, in particular with respect to elastic deformations, high adhesive strength and being as freely structurable as possible in 3 dimensions. A further object of the invention is to provide the above-mentioned coating, wherein the substrate is composed of a plastic, in particular a polymer. A further object of the invention is to provide the above-mentioned coating, wherein the coating is biocompatible. A further object of the invention is to provide an electrical device or an electrical component or both containing the above-mentioned coating. Furthermore, an object of the invention is to provide the above-mentioned coating, wherein the coating is a multiphase electrical conductor. A further object of the invention is to provide a 3D printer for producing the above-mentioned coating.

A contribution towards at least partial achievement of at least one of the above objects is provided by the independent claims. The dependent claims provide preferred embodiments that contribute towards at least partially achieving at least one of the objects.

A contribution towards achieving at least one of the objects of the invention is provided by an embodiment 1 of a composite, containing as mutually superimposed layers of a series of layers
a) a substrate, and
b) a first layer;
   wherein the first layer contains
i) a first layer surface,
ii) a polymer, and
iii) a plurality of electrically conductive particles;
   wherein the first layer surface is adjacent to the substrate; wherein at least in a first region, the first layer at a first distance from the first layer surface is characterized by a first content of the electrically conductive particles; wherein at least in the first region, the first layer at a further distance from the first layer surface is characterized by a further content of the electrically conductive particles; wherein the first content is less than the further content; wherein the first distance is less than the further distance. Preferably, the first content is less by a value in a range of 0.1 to 95% than the further content. In this case, the first distance is preferably less by a value in a range of 0.05 to 1000 μm than the further distance. More preferably, the first content is less by a value in a range of 10 to 90% than the further content. In this case, the first distance is more preferably less by a value in a range of 0.1 to 500 μm than the further distance. Most preferably, the first content is less by a value in a range of 20 to 80% than the further content. In this case, the first distance is most preferably less by a value in a range of 1 to 250 μm than the further distance.

An embodiment 2 of the composite according to the invention is configured according to embodiment 1, wherein at least in the first region, the first layer at a second distance from the first layer surface is characterized by a second content of the electrically conductive particles; wherein the second content is less than the further content and more than the first content; wherein the second distance is less than the further distance and more than the first distance. Preferably, the second content is less by at least 0.1%, more preferably by at least 20% than the further content and more than the first content. The second distance is preferably less by a value in a range of 0.05 to 1000 μm, preferably 0.1 to 500 μm, more preferably 1 to 250 μm, than the further distance and more than the first distance.

An embodiment 3 of the composite according to the invention is configured according to embodiment 1 or 2, wherein at least in the first region, the first layer at each ith distance from the first layer surface is characterized by an ith content of the electrically conductive particles; wherein each ith content is less than the (i+1)th content and more than the (i−1)th content; wherein the ith distance is less than the (i+1)th distance and more than the (i−1)th distance; wherein the nth content is less than the further content and more than the (n−1)th content; wherein the nth distance is less than the further distance and more than the (n−1)th distance; wherein i is an index in the range of 2 to (n−1), and wherein n is a natural number greater than 2. Preferably, each ith content is less by 0.1%, more preferably by at least 20%, than the (i+1)th content and more than the (i−1)th content. The ith distance is preferably less by a value in a range of 0.05 to 1000 μm, preferably 0.1 to 500 μm, more preferably 1 to 250 μm, than the (i+1)th distance and more than the (i−1)th distance.

An embodiment 4 of the composite according to the invention is configured according to one of the previous embodiments, wherein the first layer in the first region is characterized in that a content of the electrically conductive particles increases from the first distance to the further distance, preferably monotonically. This means that the content of the electrically conductive particles preferably does not decrease in any partial section from the first distance to the further distance, but can remain constant in one or a plurality of partial sections. The content of the electrically conductive particles preferably increases in a stepwise manner or continuously from the first distance to the further distance.

An embodiment 5 of the composite according to the invention is configured according to one of the previous embodiments, wherein the first layer further contains a further layer surface opposite the first layer surface, wherein the first layer in the first region is characterized in that a content of the electrically conductive particles is a monotonically increasing function of a distance from the first layer surface along a straight line from the first layer surface to the further layer surface. Here, the function is not necessarily strictly monotonically increasing.

An embodiment 6 of the composite according to the invention is configured according to one of the previous embodiments, wherein the first layer on the first layer surface is characterized by a content of the electrically conductive particles in a range of 0 to 20%, preferably 0 to 10%, more preferably 0 to 5%, based in each case on the first layer surface.

An embodiment 7 of the composite according to the invention is configured according to one of the previous embodiments, wherein the electrically conductive particles are composed of a substance selected from the group of gold, silver, palladium, platinum, and carbon or a combination of at least two thereof.

An embodiment 8 of the composite according to the invention is configured according to one of the previous embodiments, wherein the electrically conductive particles are characterized by a length in a range of 1 to 1000 μm, preferably 5 to 500 μm, more preferably 10 to 100 μm.

An embodiment 9 of the composite according to the invention is configured according to one of embodiments 1 through 8, wherein the electrically conductive particles are characterized by a diameter in a range of 0.1 to 1000 nm, preferably 0.5 to 500 nm, more preferably 1 to 100 nm.

An embodiment 10 of the composite according to the invention is configured according to one of the previous embodiments, wherein the polymer is selected from the group composed of silicone, an electrically conductive polymer, a lacquer, a polyaromatic, a thermoplastic and a resin, or a combination of at least two thereof.

An embodiment 11 of the composite according to the invention is configured according to one of the previous embodiments, wherein the composite is flexible according to the test method described herein.

An embodiment 12 of the composite according to the invention is configured according to one of the previous embodiments, wherein the first layer is characterized by
a) a contact resistance for an electrical contact to the substrate in a range of 0.1Ω to 20 kΩ, preferably 0.1Ω to 10 kΩ, more preferably 0.1Ω to 5 kΩ, most preferably 0.1Ω to 1 kΩ, or
b) a total thickness in a range of 1 to 3000 μm, preferably 1 to 2000 μm, more preferably 1 to 1500 μm, most preferably 1 to 500 μm, or
c) both.

An embodiment 13 of the composite according to the invention is configured according to one of the previous embodiments, wherein the composite further contains an additional layer, wherein the additional layer
a) is at least partially superimposed on the first layer,
b) contains an additional first layer surface,
c) contains the polymer,
d) contains an additional plurality of the electrically conductive particles;
   wherein the additional first layer surface is adjacent to the first layer, wherein at least in a further region of the additional layer, the additional layer at an additional first distance from the additional first layer surface is characterized by an additional first content of the electrically conductive particles; wherein at least in the further region, the additional layer at an additional further distance from the additional first layer surface is characterized by an additional further content of the electrically conductive particles; wherein the additional first content is more than the additional further content; wherein the additional first distance is less than the additional further distance. Preferably, the additional first content is more by a value in a range of 0.1 to 95% than the additional further content. In this case, the additional first distance is preferably less by a value in a range of 0.05 to 1000 μm than the further distance. More preferably, the additional first content is more by a value in a range of 10 to 90% than the additional further content. In this case, the additional first distance is more preferably less by a value in a range of 0.1 to 500 μm than the additional further distance. Most preferably, the additional first content is more by a value in a range of 20 to 80% than the additional further content. In this case, the additional first distance is most preferably less by a value in a range of 1 to 250 μm than the additional further distance.

An embodiment 14 of the composite according to the invention is configured according to embodiment 13, wherein at least in the further region, the additional layer at an additional second distance from the additional first layer surface is characterized by an additional second content of the electrically conductive particles; wherein the additional second content is more than the additional further content and is less than the additional first content; wherein the additional second distance is less than the additional further distance and more than the additional first distance. Preferably, the additional second content is more by at least 0.1%, more preferably by at least 20% than the additional further content and less than the additional first content. The additional second distance is preferably less by a value in a range of 0.05 to 1000 μm, preferably 0.1 to 500 μm, more preferably 1 to 250 μm, than the additional further distance and more than the additional first distance.

An embodiment 15 of the composite according to the invention is configured according to embodiment 13 or 14, wherein at least in the further region, the additional layer at each additional jth distance from the additional first layer surface is characterized by an additional jth content of the electrically conductive particles; wherein each additional jth content is more than the additional (j+1)th content and is less than the additional (j−1)th content; wherein the additional jth distance is less than the additional (j+1)th distance and more than the additional (j−1)th distance; wherein the additional mth content is more than the additional further content and is less than the additional (m−1)th content; wherein the additional mth distance is less than the additional further distance and more than the additional (m−1)th distance; wherein j is an index in the range of 2 to (m−1), wherein m is a natural number greater than 2. Preferably, each additional jth content is more by at least 0.1%, more preferably by at least 20%, than the additional (j+1)th content and less than the additional (j−1)th content. The additional jth distance is preferably less by a value in a range of 0.05 to 1000 μm than the additional (j+1)th distance and more than the additional (j−1)th distance. Preferably, each additional mth content is more by at least 0.1%, more preferably by at least 20%, than the additional further content and less than the additional (m−1)th content. The additional mth distance is preferably less by a value in a range of 0.05 to 1000 μm, preferably 0.1 to 500 μm, more preferably 1 to 250 μm, than the additional further distance and more than the additional (m−1)th distance.

An embodiment 16 of the composite according to the invention is configured according to one of embodiments 13 through 15, wherein the additional layer, at least in the further region, is characterized in that a content of the electrically conductive particles decreases from the additional first distance to the additional further distance, preferably monotonically. This means that the content of the electrically conductive particles preferably does not increase in any partial section from the additional first distance to the additional further distance, but can remain constant in one or a plurality of partial sections. Preferably, the content of the electrically conductive particles decreases in a stepwise manner or continuously from the first distance to the further distance.

An embodiment 17 of the composite according to the invention is configured according to one of embodiments 13 through 16, wherein the additional layer further contains an additional further layer surface opposite the additional first layer surface, wherein the additional layer, at least in the further region, is characterized in that a content of the electrically conductive particles is a monotonically decreasing function of a distance from the additional first layer surface along a straight line from the additional first layer surface to the additional further layer surface. Here, the function is not necessarily strictly monotonically decreasing.

An embodiment 18 of the composite according to the invention is configured according to one of embodiments 13 through 17, wherein the additional layer further contains an additional further layer surface opposite the additional first layer surface, wherein the additional layer on the additional further layer surface is characterized by a content of the electrically conductive particles in a range of 0 to 20%, preferably 0 to 10%, more preferably 0 to 5%, based in each case on the additional further layer surface.

An embodiment 19 of the composite according to the invention is configured according to one of embodiments 13 through 18, wherein the additional layer further contains an additional further layer surface opposite the additional first layer surface, wherein the additional layer on the additional further layer surface has a specific electrical conductivity of less than 6500 S/m, preferably less than 6000 S/m, more preferably less than 5500 S/m, most preferably less than 5000 S/m. Preferably, the additional layer is electrically insulating on the additional further layer surface.

An embodiment 20 of the composite according to the invention is configured according to one of embodiments 13 through 19, wherein the electrically conductive particles of the additional plurality of electrically conductive particles are composed of a substance selected from the group of gold, silver, palladium, platinum, and carbon or a combination of at least two thereof.

An embodiment 21 of the composite according to the invention composite is configured according to one of embodiments 13 through 20, wherein the electrically conductive particles of the additional plurality of electrically conductive particles are characterized by a length in a range of 1 to 1000 μm, preferably 5 to 500 μm, more preferably 10 to 100 μm.

An embodiment 22 of the composite according to the invention is configured according to one of embodiments 13 through 21, wherein the electrically conductive particles of the additional plurality of electrically conductive particles are characterized by a diameter in a range of 0.1 to 1000 nm, preferably 0.5 to 500 nm, more preferably 1 to 100 nm.

An embodiment 23 of the composite according to the invention is configured according to one of embodiments 13 through 22, wherein the additional layer is superimposed on the first layer in a first partial area of the first layer, wherein a contacting layer is superimposed on the first layer in at least one further partial area of the first layer, wherein the contacting layer on a surface of the contacting layer facing away from the first layer is characterized by a contact resistance in a range of 0.1Ω to 20 kΩ, preferably 0.1Ω to 10 kΩ more preferably 0.1Ω to 5 kΩ, most preferably 0.1Ω to 1 kΩ. Preferably, the contacting layer is superimposed on the first layer in 2 partial areas separated from each other by the additional layer. The contacting layer therefore preferably forms at least 2 electrodes by means of which an electrical contact can be established. A preferred contacting layer forms an electrical contact. Preferably, the contacting layer forms two electrical contacts separated from each other by the additional layer. The contacting layer preferably contains the electrically conductive particles to a content in a range of 1 to 100%, more preferably 1 to 50%, more preferably 1 to 30%, more preferably 5 to 30%, further preferably 5 to 25%, based in each case on the surface of the contacting layer facing away from the first layer. Moreover, the contacting layer preferably contains the polymer.

An embodiment 24 of the composite according to the invention is configured according to one of embodiments 13 through 23, wherein the first partial area is adjacent to the further partial area.

An embodiment 25 of the composite according to the invention is configured according to one of the preceding embodiments, wherein the substrate is composed of a substance selected from the group of a plastic, a plastic mixture, and a metal, or a combination of at least two thereof.

An embodiment 26 of the composite according to the invention is configured according to one of the preceding embodiments, wherein the substrate is contained by one selected from the group composed of a medical device, a medical aid and an electrical device or a combination of at least two thereof.

An embodiment 27 of the composite according to the invention is configured according to one of the preceding embodiments, wherein the substrate is selected from the group composed of a tube, a catheter, a wire, a needle, a probe, an implant, a film, a cannula and a lead, or a combination of at least two thereof.

An embodiment 28 of the composite according to the invention is configured according to one of the preceding embodiments, wherein the composite is selected from the group composed of a medical device, a medical aid, a plug and a socket or a combination of at least two thereof.

A contribution towards achieving at least one of the objects of the invention is provided by an embodiment 1 of an apparatus 1, containing a substrate and a coating, wherein the substrate contains a substrate surface; wherein the coating
a) contains a first surface and a further surface,
   wherein the first surface is adjacent to the substrate surface,
   wherein the further surface faces away from the substrate;
b) contains a polymer and a pluarlity of electrically conductive particles;
c) is characterized on the first surface by a content of the electrically conductive particles in a range of 0 to 20%, preferably 0 to 10%, more preferably 0 to 5%, based in each case on the first surface;
d) contains a first partial volume; and
e) in the first partial volume, is characterized by a content of the electrically conductive particles in a range of 1 to 100 vol.-%, preferably 1 to 50 vol.-%, more preferably 1 to 30 vol.-%, more preferably 5 to 30 vol.-%, further preferably 5 to 25 vol.-%, based in each case on the volume of the coating in the first partial volume;

wherein in a first region of the coating
i) the coating is characterized in that along a straight line from the first surface to the further surface, a content of the electrically conductive particles in the coating is a function of a position on the straight line with at least one first local maximum,
   wherein the first local maximum is contained by the first partial volume,
   wherein the function of the first local maximum decreases respectively to one adjacent minimum in the direction of the first surface and one adjacent minimum in the direction of the further surface continuously or in at least 2 steps, preferably in at least 3 steps, more preferably in at least 4 steps, most preferably in at least 5 steps; and
ii) the coating on the further surface is characterized by a content of the electrically conductive particles in a range of 0 to 20%, preferably 0 to 10%, more preferably 0 to 5%, based in each case on the further surface.

An embodiment 2 according to the invention of the apparatus 1 is configured according to embodiment 1, wherein in the first region, the first partial volume is longitudinally extended or sheetlike. A volume is longitudinally extended if it is more extended in one dimension by at least a factor of 2, preferably at least a factor of 3, more preferably by at least a factor of 10, than it is in the two other dimensions, perpendicular to the first dimension in each case. A volume is sheetlike if it is more extended in two dimensions by at least a factor of 2, preferably at least a factor of 3, more preferably by at least a factor of 10, than it is in the other dimension, perpendicular to each of the two dimensions. A preferred sheetlike volume is curved, preferably curved along a jacket surface of a cylinder.

An embodiment 3 according to the invention of the apparatus 1 is configured according to embodiments 1 or 2, wherein the coating contains a further region, wherein the first partial volume in the further region contains the further surface of the coating. Preferably, the further surface in the further region is a contacting surface with a contact resistance in a range of 0.1Ω to 20 kΩ, preferably 0.1Ω to 10 kΩ, more preferably 0.1Ω to 5 kΩ, most preferably 0.1Ω to 1 kΩ. In this embodiment, the first partial volume can thus be electrically contacted from outside the coating on the part of the further surface that is contained by the first partial volume. Accordingly, the first partial volume can function as an electrical conductor in the coating, to which an electrical contact can be established in the further region via the further surface.

An embodiment 4 according to the invention of the apparatus 1 is configured according to one of embodiments 1 through 3, wherein the coating
a) contains at least one further partial volume,
b) is characterized in the further partial volume by a content of the electrically conductive particles in a range of 1 to 100 vol.-%, preferably 1 to 50 vol.-%, more preferably 1 to 30 vol.-%, more preferably 5 to 30 vol.-%, further preferably 5 to 25 vol.-%, based in each case on the volume of the coating in the further partial volume;
   wherein in the first region of the coating, the function contains at least one further local maximum, wherein the further local maximum is contained by the further partial volume, wherein the function decreases continuously or in at least 2 steps, preferably in at least 3 steps, more preferably in at least 4 steps, most preferably in at least 5 steps, from the further local maximum to one adjacent minimum in the direction of the first surface and one adjacent minimum in the direction of the further surface respectively, wherein the further partial volume is electrically insulated from the first partial volume. Preferably, the first partial volume and the further partial volume each form one phase of a multiphase electrical conductor.

An embodiment 5 according to the invention of the apparatus 1 is configured according to one of embodiments 1 through 4, wherein the first partial volume or the further partial volume or both respectively is/are (an) electrode(s). A preferred electrode is a ring electrode.

An embodiment 6 according to the invention of the apparatus 1 is configured according to one of embodiments 1 through 5, wherein the substrate with the coating is flexible according to the test method described herein.

An embodiment 7 according to the invention of the apparatus 1 is configured according to one of embodiments 1 through 6, wherein the substrate is contained in one selected from the group composed of a medical device, a medical aid and an electrical device or a combination of at least two thereof.

An embodiment 8 according to the invention of the apparatus 1 is configured according to one of embodiments 1 through 7, wherein the substrate is selected from the group composed of a catheter, a wire, a needle, a probe, an implant, a film, a cannula and a lead, or a combination of at least two thereof.

An embodiment 9 according to the invention of the apparatus 1 is configured according to one of embodiments 1 through 8, wherein the apparatus is selected from the group composed of a medical device, a medical aid, a plug and a socket or a combination of at least two thereof.

An embodiment 10 according to the invention of the apparatus 1 is configured according to one of embodiments 1 through 9, wherein the electrically conductive particles are composed of a substance selected from the group of gold, silver, palladium, platinum, and carbon or a combination of at least two thereof.

An embodiment 11 according to the invention of the apparatus 1 is configured according to one of embodiments 1 through 10, wherein the electrically conductive particles are characterized by a length in a range of 1 to 1000 μm, preferably 5 to 500 μm, more preferably 10 to 100 μm.

An embodiment 12 according to the invention of the apparatus 1 is configured according to one of embodiments 1 to 11, wherein the electrically conductive particles are characterized by a diameter in a range of 0.1 to 1000 nm, preferably 0.5 to 500 nm, more preferably 1 to 100 nm.

An embodiment 13 according to the invention of the apparatus 1 is configured according to one of embodiments 1 through 12, wherein the polymer is selected from the group composed of silicone, an electrically conductive polymer, a lacquer, a polyaromatic, a thermoplastic and a resin, or a combination of at least two thereof.

An embodiment 14 according to the invention of the apparatus 1 is configured according to one of embodiments 1 through 13, wherein the coating is characterized by
a) a contact resistance between the substrate surface and the first surface in a range of 0.1Ω to 20 kΩ, preferably 0.1Ω to 10 kΩ, more preferably 0.1Ω to 5 kΩ, most preferably 0.1Ω to 1 kΩ, or
b) a total thickness in a range of 8 μm to 1 cm, preferably 10 μm to 5 mm, more preferably 50 μm to 1 mm, or
c) both.

A contribution towards achieving at least one of the objects of the invention is provided by an embodiment 1 of a method, containing as method steps
a) provision of a substrate and n compositions;
   wherein the substrate contains a substrate surface,
   wherein each of the n compositions contains a polymer and a plurality of electrically conductive particles in a particle content based on the weight of the respective composition,
   wherein the n compositions are characterized in that the respective particle contents of the n compositions differ from one another; and
b) superimposition on the substrate surface of at least one first portion each of the n compositions,
   wherein the superimposition of at least the first portions of the n compositions takes place successively, wherein after each superimposition of at least the first portion of one of the n compositions, the at least one first portion is cured, wherein at least the first portions of the n compositions are superimposed in a series of increasing particle contents, wherein a first series of layers is obtained containing from the substrate surface in a layer sequence direction a first to an nth layer of mutually superimposed layers, wherein n is a natural number greater than 1, preferably greater than 2, more preferably greater than 3, more preferably greater than 4, most preferably greater than 5. According to method step b), multiple portions of one of the n compositions can be superimposed respectively on the substrate surface in direct succession, i.e. without there being a portion of a composition with another particle content in between. This is particularly advantageous in order to produce a specified layer thickness.

An embodiment 2 of the method according to the invention is configured according to embodiment 1, wherein the method further comprises
c) superimposition on the nth layer of the first series of layers, at least in a partial area of the nth layer, of at least one further portion respectively of the (n−1)th to first composition,
   wherein the superimposition of at least the further portions of the (n−1) compositions takes place successively, wherein after each superimposition of at least the further portion of one of the (n−1) compositions, the at least one further portion is cured, wherein at least the further portions of the (n−1) compositions are superimposed in a sequence of decreasing particle contents, wherein a second series of layers is obtained containing, as mutually superimposed layers from the nth layer in the layer sequence direction, a further (n−1)th to a further first layer. According to method step c), multiple further portions of one of the (n−1)th to first compositions can be superimposed respectively on the nth layer of the first series of layers in direct succession, i.e. without there being a further portion of a composition with another particle content in between. This is particularly advantageous in order to produce a specified layer thickness.

An embodiment 3 of the method according to the invention is configured according to embodiment 2, wherein in a further method step d), a surface of the further first layer is electrically deactivated. The above-mentioned electrical deactivation is preferably carried out if the polymer is an electrically conductive polymer, particularly preferably PEDOT. Preferably, the surface of the further first layer is electrically deactivated by etching.

An embodiment 4 of the method according to the invention is configured according to one of embodiments 1 through 3, wherein the curing of the first portions or the curing of the further portions or both is carried out by irradiation with light or heating, or both. A preferred type of irradiation with light is irradiation with infrared light or ultraviolet light, or both.

An embodiment 5 of the method according to the invention is configured according to embodiment 4, wherein heating is carried out to a temperature in a range of 50 to 300° C., preferably 50 to 250° C., more preferably 70 to 200° C., more preferably 80 to 150° C.

An embodiment 6 of the method according to the invention is configured according to one of embodiments 1 through 5, wherein the method is an additive production method. A preferred additive production method is 3D printing. The additive production method is to be distinguished from a subtractive production method. A further preferred additive production method is selected from the group composed of a powder bed method, a free space method and a liquid material method or a combination of at least two thereof. A particularly preferred free space method is fused deposition modeling (FDM).

An embodiment 7 of the method according to the invention is configured according to one of embodiments 1 through 6, wherein the method is carried out with a 3D printer.

An embodiment 8 of the method according to the invention is configured according to one of embodiments 1 through 7, wherein the superimposition in method step b) or c) or in both is carried out by application with a nozzle, wherein the nozzle contains a nozzle opening with a diameter in a range of 100 nm to 2000 μm, preferably 200 nm to 1000 μm, more preferably 300 nm to 500 μm.

An embodiment 9 of the method according to the invention is configured according to one of embodiments 1 through 6, wherein the superimposition in method step b) or c) or in both takes place by immersion. Here, the immersion in method step b) preferably takes place into one of the n compositions each. Here, the substrate surface is preferably immersed and wetted with a portion of a composition. The portion is cured and a first layer is thus obtained. A surface of the first layer is immersed in the same composition or in another composition with a greater particle content and thus wetted with a portion of the composition. This portion is in turn cured, and a second layer is thus obtained. The first to nth layers are obtained in this manner.

In method step c), immersion is preferably carried out in one of the (n−1)th to first compositions each time.

A contribution towards achieving at least one of the objects of the invention is provided by an embodiment 1 of an apparatus 2, obtainable by the method according to one of embodiments 1 through 9.

A contribution towards achieving at least one of the objects of the invention is provided by an embodiment 1 of an electrical component containing the composite according to one of its embodiments 1 through 28; or the apparatus 1 according to one of its embodiments 1 through 14; or the apparatus 2 according to its embodiment 1.

A contribution towards achieving at least one of the objects of the invention is provided by an embodiment 1 of an electrical device containing the composite according to one of its embodiments 1 through 28; or the apparatus 1 according to one of its embodiments 1 through 14; or the apparatus 2 according to its embodiment 1; or the electrical component according to its embodiment 1.

An embodiment 2 of the electrical device according to the invention is configured according to its embodiment 1, wherein the electrical device further contains a sensor.

A contribution towards achieving at least one of the objects of the invention is provided by an embodiment 1 of a 3D printer configured to produce the composite according to one of its embodiments 1 through 28; or the apparatus 1 according to one of its embodiments 1 through 14; or the apparatus 2 according to its embodiment 1. The 3D printer preferably contains a nozzle with a nozzle opening having a diameter in a range of 100 nm to 2000 μm, preferably 200 nm to 1000 μm, more preferably 300 nm to 500 μm.

A contribution towards achieving at least one of the objects of the invention is provided by an embodiment 1 of a use of a composition containing a polymer and a plurality of electrically conductive particles for electrical contacting of a coating superimposed on a substrate.

Preferred embodiments of components of a category according to the invention, in particular of the composite, the apparatus and the method, are equally preferred for components of the other categories according to the invention that have the same names or are corresponding components.

In this document, the statement that an entity is superposed on another means that the former entity follows the latter. Here, the entity can follow the other directly or indirectly. Accordingly, according to this term, the two entities can either be in contact with each other or not. In contrast, the statement that an entity is adjacent to another means that the two entities are in direct contact with each other. Here, an entity is typically a layer, a substrate or a region.

### Polymer

A preferred polymer is an electrically conductive polymer. A preferred electrically conductive polymer is polyaniline or a polyoxythiophene, or both. A preferred polyoxythiophene is poly-3,4-ethylenedioxythiophene (PEDOT). A preferred PEDOT is poly-3,4-ethylenedioxythiophene-polystyrene sulfonate (PEDOT:PSS). A preferred electrically conductive polymer is characterized by a lower specific electrical conductivity than the particles of the plurality of electrically conductive particles, preferably by at least 1·10⁶ S/m, more preferably by at least 10·10⁶ S/m, most preferably by at least 50·10⁶ S/m.

Another preferred polymer is a non-electrically conductive polymer. Here, particularly suitable examples include duroplastic, thermoplastic, and elastic polymers. Duroplastic polymers melt only poorly and—if at all—at a temperature of over 250° C. A preferred duroplastic polymer is a resin, in particular an epoxy resin, a polyaromatic, preferably parylene, or polyurethane resin, wherein a polyaromatic, preferably parylene, is particularly preferred. A preferred thermoplastic is one selected from the group composed of a polycarbonate (PC), a polyethylene (PE), and polymethyl methacrylate (PMMA), or a combination of at least two thereof. A preferred polyethylene is polytetrafluoroethylene (PTFE). A preferred elastic polymer is a latex, a rubber and particularly preferably a silicone-based polymer. The polymer can also be in the form of a lacquer. A preferred lacquer is a photoresist. A preferred photoresist is a negative resist or a positive resist, or both. A preferred negative resist is SU-8.

### Region

In the context of the composite according to the invention, a region of a layer preferably refers a laterally flat section of the layer in its entire thickness. Preferably, the further region of the additional layer is at least partially superimposed on the first region of the first layer.

### Electrically Conductive Particles

Preferred electrically conductive particles do not contain the polymer. Further preferred electrically conductive particles are metal particles or carbon particles, or both. Preferred carbon particles are carbon tubes, preferably carbon nanotubes. Preferred metal particles are wires, preferably silver or gold wires. Further preferred electrically conductive particles are selected from the group composed of spherical, plate-shaped, rod-shaped, tube-shaped, cube-shaped, cuboid-shaped, and needle-shaped particles or a combination of at least two thereof. Further preferred electrically conductive particles are longitudinally extended. Preferred longitudinally extended electrically conductive particles are selected from the group composed of tubeshaped, prism-shaped, rod-shaped, wire-shaped, and thread-shaped particles or a combination of at least two thereof. Further preferred carbon particles contain, and are more preferably composed of, graphene or carbon black, or both.

### Substrate

A preferred plastic, of which the substrate is preferably composed, is a non-conductive polymer. This polymer is preferably selected from the group composed of silicone, a polyaromatic, a thermoplastic and a resin, or a combination of at least two thereof. A more preferable polyaromatic is a parylene. A preferred resin is an epoxy resin. A preferred thermoplastic is one selected from the group composed of a polycarbonate (PC), a polyethylene (PE), and polymethyl methacrylate (PMMA), or a combination of at least two thereof. A preferred polyethylene is polytetrafluoroethylene (PTFE). Particularly preferred for a substrate are a polyurethane, a polyaromatic, in particular parylene, a silicone, or a combination of at least two thereof. A preferred substrate surface is flat or curved, or both. A preferred curved substrate surface is at least partially a jacket surface of a cylinder.

### Composition

A preferred composition is a suspension or a paste, or both. A preferred paste is characterized by a viscosity in a range of 0.001 to 10⁸ Pa·s, more preferably 0.001 to 10⁷ Pa·s, most preferably 0.001 to 10⁶ Pa·s. The suspension or paste further contains a diluent. The diluent is selected such that it evaporates at a temperature in a range of 40 to 500° C., preferably in a range of 50 to 200° C. and particularly preferably in a range of 60 to 150° C. Particularly preferred diluents are surfactants. These can be ionic or nonionic, with nonionic surfactants being particularly preferred. Nonionic surfactants are commercially available, for example under the brand names Tween® or Triton®. The amount of the diluent is selected depending on the further components of the composition such that the respectively desired viscosity of the composition is adjusted. In addition to the polymer and the plurality of electrically conductive particles, a preferred composition further contains diluents. In this case, the composition with the lowest content of electrically conductive particles preferably contains these in a content in a range of 0 to 60 wt.-%, more preferably 0 to 50 wt.-%, most preferably 0 to 40 wt.-%, based in each case on the weight of the composition. Moreover, the composition with the highest content of electrically conductive particles preferably contains these in a content in a range of 20 to 95 wt.-%, more preferably 30 to 90 wt.-%, most preferably 40 to 85 wt.-%, based in each case on the weight of the composition.

### Electrical Deactivation

Preferred electrical deactivation is carried out by means of contact with a halogen or a halogen-containing compound, or both. In this case, the surface to be electrically deactivated is preferably partially halogenated.

### Medical Device

A medical device is configured to carry out treatment of a disease or disorder; or a diagnostic procedure; or both. A preferred medical device is electrically operated. A further preferred medical device is an implant. A preferred implant is selected from the group composed of a pacemaker, a biomonitor and a neurostimulator or a combination of at least two thereof. Here, the substrate is particularly preferably contained by a lead of the pacemaker. Moreover, a pacemaker contains an electrical pulse generator.

### Medical Aid

A medical aid is designed to be used in medical treatment of the human or animal body. A preferred medical treatment is selected from the group composed of a therapy, an intervention and a diagnostic examination. A preferred intervention comprises removal from the human or animal body or insertion into the human or animal body. A preferred removal is the taking of a sample. A preferred sample is a blood sample or a tissue sample. A preferred insertion is an implantation. A further preferred intervention is a surgical intervention. A preferred medical aid is selected from the group composed of a needle, a cannula, a catheter and a bag or a combination of at least two thereof. An preferred bag is a drip bag or a blood bag, or both.

### Electrical Component

An electrical component is the basic component of an electrical circuit and is considered as a unit. A preferred electrical component is selected from the group composed of a passive component, an active component, a linear component, a non-linear component, a discrete component and an integrated component or a combination of at least two thereof. A preferred passive component is selected from the group composed of a resistor, an inductor and a capacitor or a combination of at least two thereof. A preferred active component is a transistor or a relay, or both.

### Electrical Device

An electrical device is a device operated by means of electrical energy for private or commercial use. Using electricity, therefore, one or a plurality of objects can be achieved. The electrical device is selected from the group composed of a device that can be directly supplied with energy from the power grid, a device equipped with at least one accumulator and a device equipped with at least one battery, or a combination of at least two thereof. The electrical device contains one or a plurality of electrical components.

### MEASUREMENT METHODS

The following measurement methods were used in the context of the invention. Unless otherwise indicated, the measurements were carried out at an ambient temperature of 25° C., an ambient pressure of 100 kPa (0.986 atm) and a relative humidity of 50%.

### Particle Content

A section through the series of layers is first made with a scalpel. Here, the section is made at the distance from the surface of the series of layers at which the particle content is to be determined. A surface of the series of layers is thus exposed that is to be examined for its particle content. Here, the distance of the measurement position from the layer surface or the substrate is determined on the section under a light microscope (with an EPIPLAN 10 lens from Carl Zeiss Microscopy GmbH). Determination of the particle content is carried out on the exposed section.

An image of the surface to be examined is first prepared with a scanning electron microscope (SEM). For this purpose, the SEM is operated in secondary electron contrast mode (SE) with an acceleration voltage of 8 kV. The image is produced with 10000× magnification and stored as a TIF file. In the image, the electrically conductive particles appear lighter than the polymer in which they are embedded. The SEM image is opened using the software ImageJ (Open Source, Version 1.51). The cursor is used to select a 400 pixels×400 pixels square section in the image. The image is cut to this section, and the section is opened for further processing. This is carried out by the following commands:

File—Edit—Cut;

File—New—Image, with the standard settings Type: 8-bit, Fill with: Black, Width: 400 pixels, Height: 400 pixels, Slices: 1 in the window “New Image . . . ”; and

File—Edit—Paste

The section is further subjected to image processing. The image is first sharpened, and brightness and contrast are then adjusted. The commands used for this purpose are as follows:

Process—Sharpen;

Process—Find edges; and

Image—Adjust—Brightness/Contrast

In the window “B&C”, the settings “Minimum”, “Maximum”, “Brightness” and “Contrast” are selected such that particles to be seen in the image section, which are exposed in this section, are displayed in white. The polymer and particles hidden therein in the depth of the image are displayed in black in the setting to be selected. In this manner, an essentially two-stage image is obtained in which the electrically conductive particles located on the section are displayed in white. This is implemented with the following commands:

Adjust;

Process—Binary—Make binary

Moreover, the two-stage image obtained is displayed as a negative so that the particles are now displayed in black. The following command is

Analyze—Measure Mean.

The software now shows the window “Results”. In this window, the total area of the image section is shown under “Area”, the mean grey value of the image section under “Mean”, the minimum grey value of the image section under “Min” and the maximum grey value of the image section under “Max”. Here, Min must=0 if the above image processing has been carried out correctly. Moreover, Max should=255. The percent content of the electrically conductive particles on the section is calculated therefrom as follows:

Particle content [%]=(Mean·100)/Max.

The above process is repeated on five SEM images of the section and as result for the particle content in percent the arithmetic mean is determined. Accordingly, the content of the electrically conductive particles is indicated based on a plane located at the distance to be investigated from the substrate or the layer surface. If the particle content is to be indicated in a volume, 3 sections through this volume are made as described above, and the particle content is determined as described above for each of the sections. The particle content of the volume is the arithmetic mean of the 3 sections through the volume and is indicated in vol.-%.

### Flexibility

In order to determine whether a substrate or a composite is flexible, a sample of the substrate or composite of sufficient length is used. Moreover, the sample should have a width of at least 1 cm. The sample is wound around a rod with a diameter of 20 mm with incomplete winding of 300°. The sample is then unwound and again wound around the rod in the opposite direction to 300°. The above sequence is repeated 3 times (winding a total of 6 times). The sample is then smoothed flat by hand on a flat base. The sample is now observed under a light microscope at 10× magnification. If no damage is observed on the surface of the sample, the tested composite is considered to be flexible within the meaning of the invention.

### Specific Electrical Conductivity

Specific electrical conductivity is determined as the inverse value of specific electrical resistance. Specific electrical resistance is determined according to the standard ISO 2878:2011(E). Measurement is carried out at a temperature of 23±2° C. and a relative humidity of 50±5%.

### Total Thickness (Layer, Coating)

The total thickness of the layer or the coating is measured using a light microscope with graduations. For this purpose, a section is made through the layer structure using a scalpel.

Contact Resistance

Two measuring cables are first connected with hook clips (set of Fluke AC280 SureGrip) to a commercially available multimeter. In the following measurements, the hook clips are attached to the sample in each case with a contact pressure of 4 MPa. First, the first hook clip is attached to a first position on the sample. The second clip is attached to the sample at a distance I₁. The electrical resistance R₁ is measured with the multimeter. The second clip is then loosened and attached to the sample in the same direction as I₁, but at a greater distance I₂ from the first position. In this case, taking into account the sample size, I₂ should be at least twice as large as I₁. The electrical resistance, now R₂, is then again measured with the multimeter. The contact resistance R_{contact} is calculated as follows:

R_{contact}=(R₂I₁−R₁I₂)/(2I₁−2I₂).

### Adhesive Strength

Adhesive strength is determined according to DIN EN ISO 2409:2013-06. Here, a cutting distance of 60 μm is selected, and a cutting knife with a rigid blade is used. 6 perpendicular sections, and deviating from the standard, as many parallel sections as possible, are prepared, but at least 2. The term vertical here means perpendicular to the direction of longitudinal extension in the case of a longitudinally extended substrate, and perpendicular to the substrate surface in the case of a sheetlike substrate.

### Biocompatibility

Biocompatibility is determined according to the standard ISO 10993-4:2002.

In the following, the invention is presented in further detail by means of examples and drawings, wherein the examples and drawings are not to be interpreted as limiting the invention.

### COMPARATIVE EXAMPLE 1

### Not According to the Invention

500 ml of Elastosil A (Wacker Silicones) is thoroughly mixed with 500 ml of Elastosil B (Wacker Silicones), 800 ml of carbon nanotubes (Nanocyl NC 7000 from Safic Alcan), and 100 ml of xylene (Diggers) in a commercially available kitchen mixer. A portion of the mixture obtained is loaded into a syringe with an opening diameter of 1 mm. The syringe is used to apply a conductor track from the mixture to a PU tube with a diameter of 150 mm (Vention: article 115-0132) from one end to the other. The conductor track on the tube is cured in a convection oven at 100° C. for 5 hours.

### COMPARATIVE EXAMPLE 2

### Not According to the Invention

Comparative example 2 is carried out in the same manner as comparative example 1, except that 100 ml of the carbon nanotubes is used.

### EXAMPLE 1

### According to the Invention

The three following compositions are prepared.

Composition 1:

500 ml Elastosil A (Wacker Silicones)

500 ml Elastisol B (Wacker Silicones)

100 ml Xylene (Diggers)

Composition 2:

500 ml Elastosil A (Wacker Silicones)

500 ml Elastisol B (Wacker Silicones)

500 ml carbon nanotubes (Nanocyl NC 7000 von Safic Alcan)

100 ml Xylene (Diggers)

Composition 3:

500 ml Elastosil A (Wacker Silicones)

500 ml Elastisol B (Wacker Silicones)

800 ml carbon nanotubes (Nanocyl NC 7000 von Safic Alcan)

100 ml Xylene (Diggers)

The above-listed components of composition 1 are thoroughly mixed in a commercially available kitchen mixer. A portion of each mixed composition 1 is loaded into a syringe with an opening diameter of 1 mm. The corresponding syringe is used to apply a track from the first mixed composition to a PU tube with a diameter of 150 mm (Vention: article 115-0132) from one end to the other. The track on the tube is cured in a convection oven at 100° C. for 5 hours. The components of the composition 2 are then thoroughly mixed analogously to the method described above for composition 1 and loaded into a syringe, and the cured track from composition 1 on the tube is then superimposed by the mixed composition 2. The applied composition 2 is in turn cured in the convection oven at 100° C. for 5 hours. The components of the composition 3 are then thoroughly mixed analogously to the method described above for composition 1 and loaded into a syringe, and the cured track from the composition 2 on the tube is superimposed by with the mixed composition 3. The applied composition 3 is in turn cured in the convection oven at 100° C. for 5 hours. A flexible tube with a conductor track having a content gradient of electrically conductive carbon nanotubes according to the invention is obtained.

Evaluation

| | | Adhesive strength on |
|---|---|---|
| | Electrical conductivity | substrate |
| Comparative example 1 | ++ | −− |
| Example 1 | + | + |
| Comparative example 2 | − | ++ |

In the above table, ++ indicates a more advantageous result than +, + a more advantageous result than −, and − a more advantageous result than −−.

Accordingly, in the example according to the invention, a flexible tube with a conductor track is obtained that shows an advantageous combination of favourable electrical conductivity and favourable adhesive strength on the substrate (the tube).

The figures show the following:

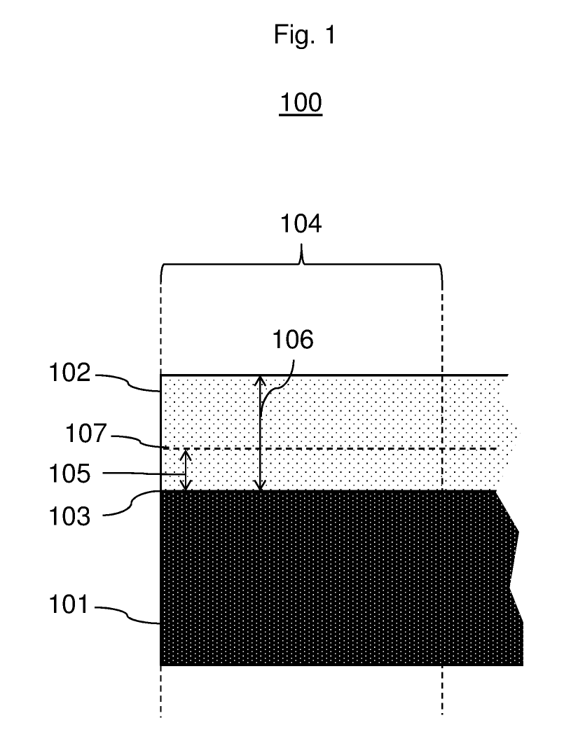

FIG. 1 a diagrammatic sectional view of a composite according to the invention;

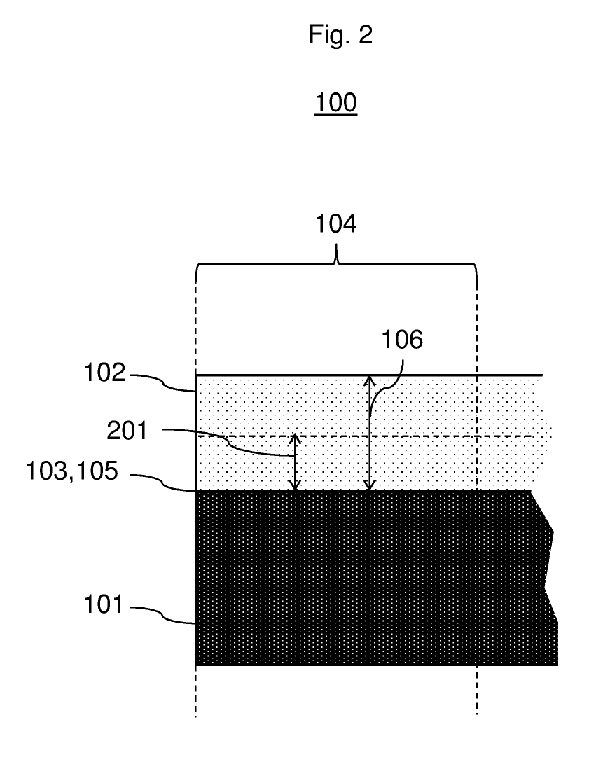

FIG. 2 a diagrammatic sectional view of a further composite according to the invention;

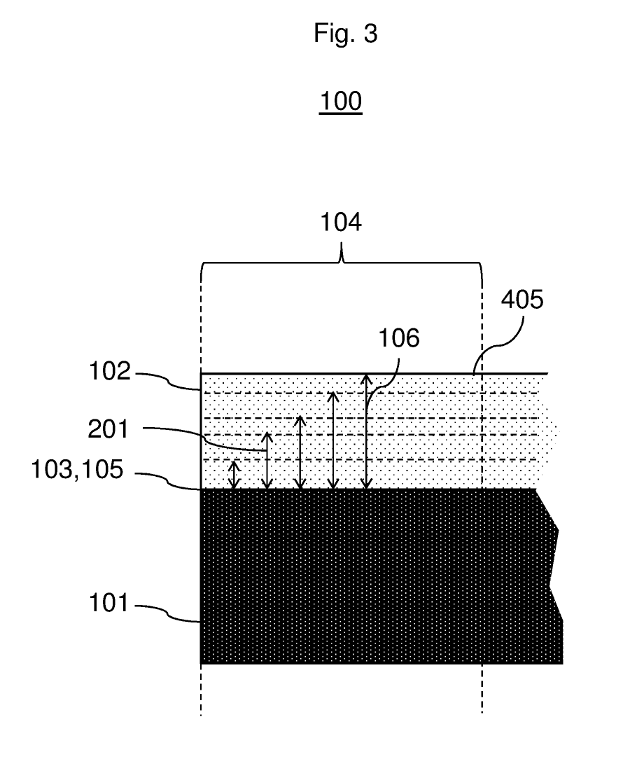

FIG. 3 a diagrammatic sectional view of a further composite according to the invention;

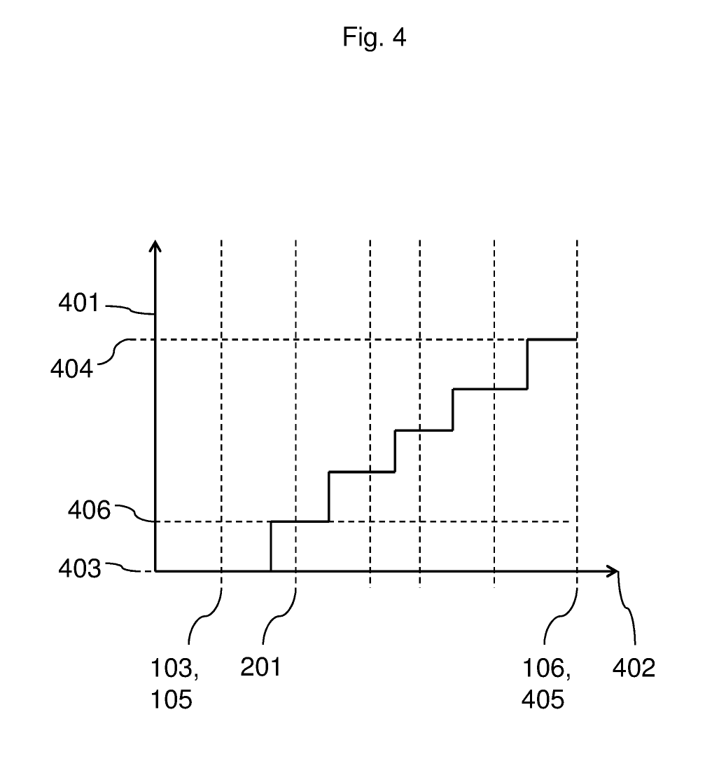

FIG. 4 a graphic representation of the content of electrically conductive particles of the composite in FIG. 3;

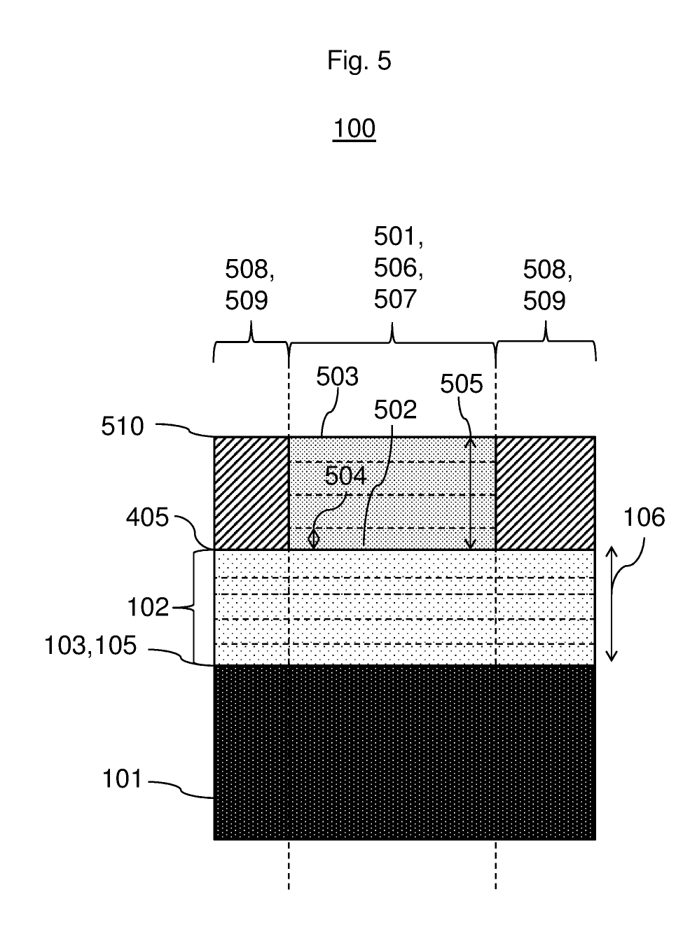

FIG. 5 a diagrammatic sectional view of a further composite according to the invention;

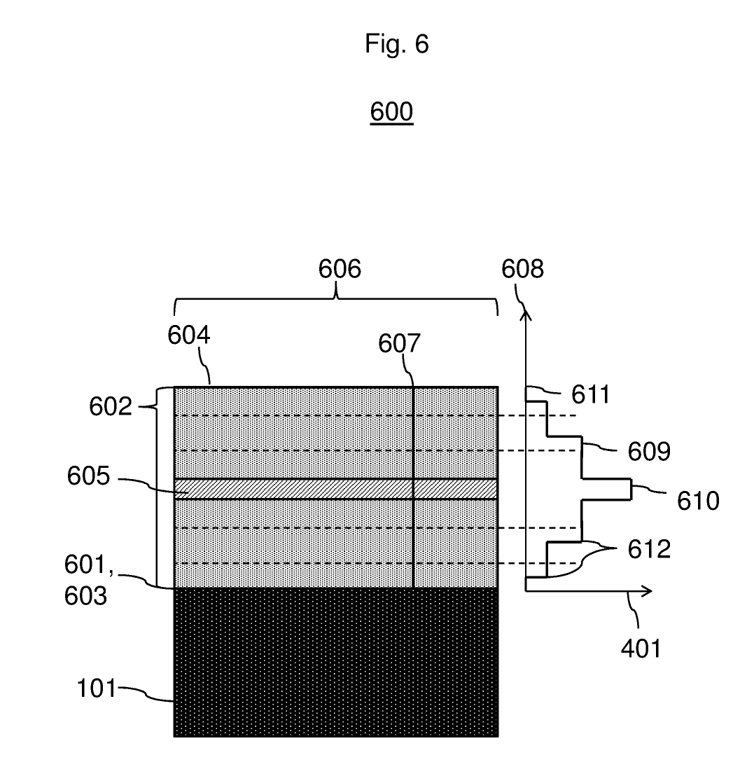

FIG. 6 a diagrammatic sectional view of an apparatus according to the invention with a graphic representation of a content of electrically conductive particles;

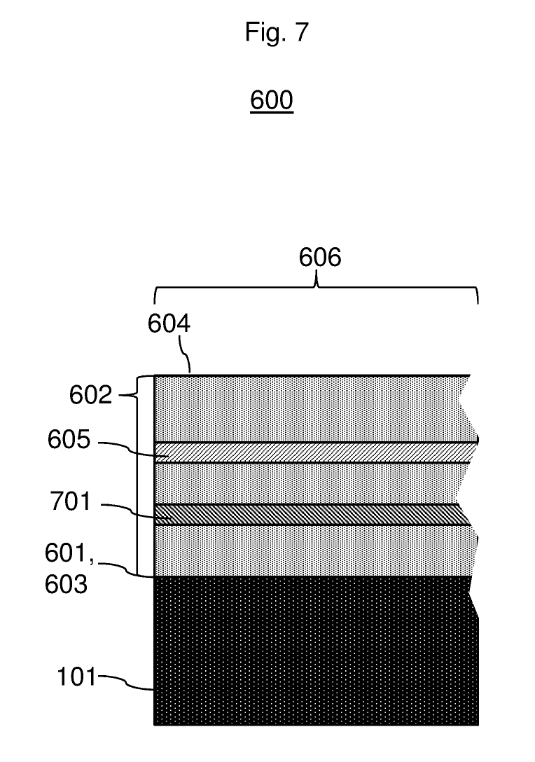

FIG. 7 a diagrammatic sectional view of a further apparatus according to the invention;

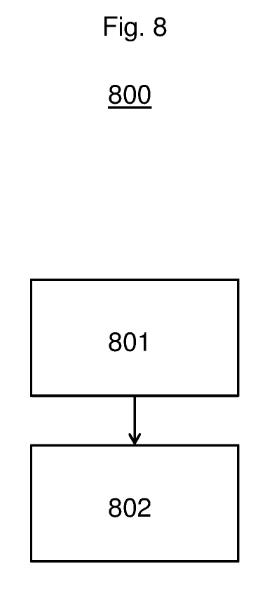

FIG. 8 a flow chart of a method according to the invention;

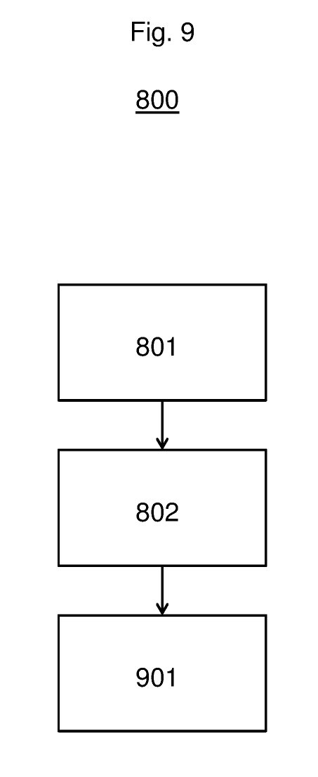

FIG. 9 a flow chart of a further method according to the invention;

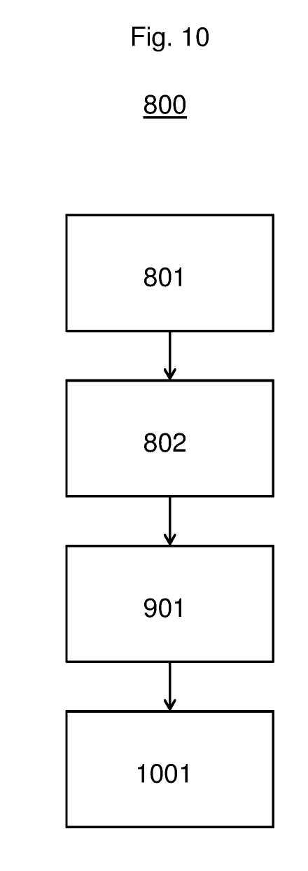

FIG. 10 a flow chart of a further method according to the invention;

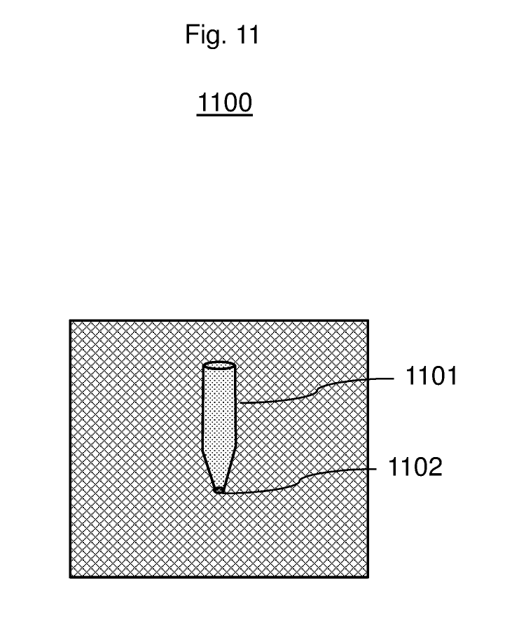

FIG. 11 a diagrammatic view of a 3D printer according to the invention;

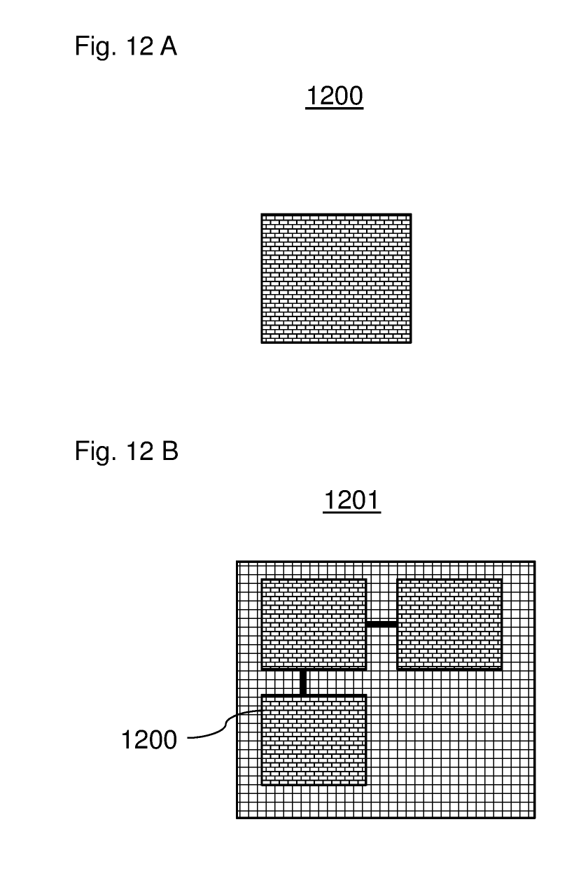

FIG. 12A a diagrammatic view of an electrical component according to the invention;

FIG. 12B a diagrammatic view of an electrical device according to the invention;

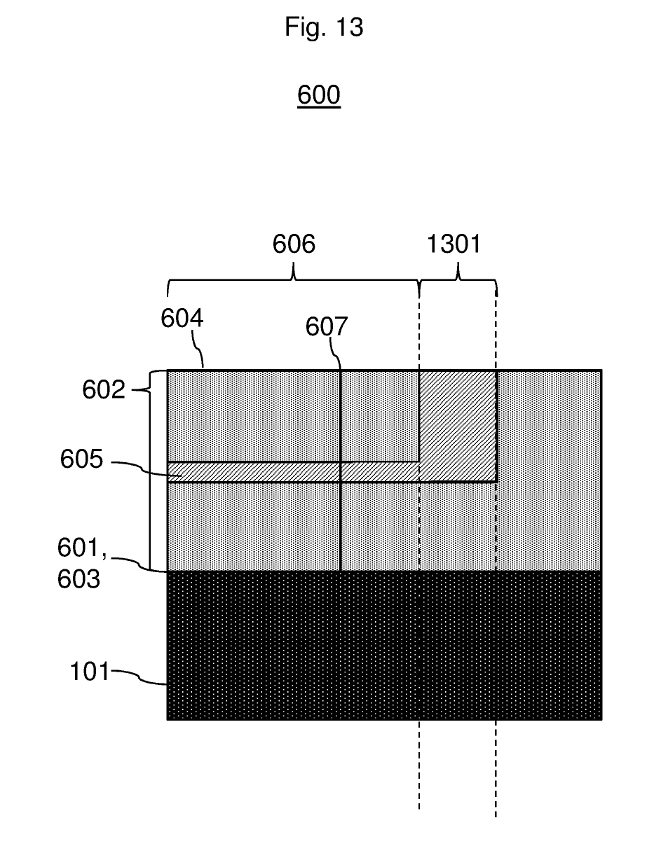

FIG. 13 a diagrammatic sectional view of a further apparatus according to the invention;

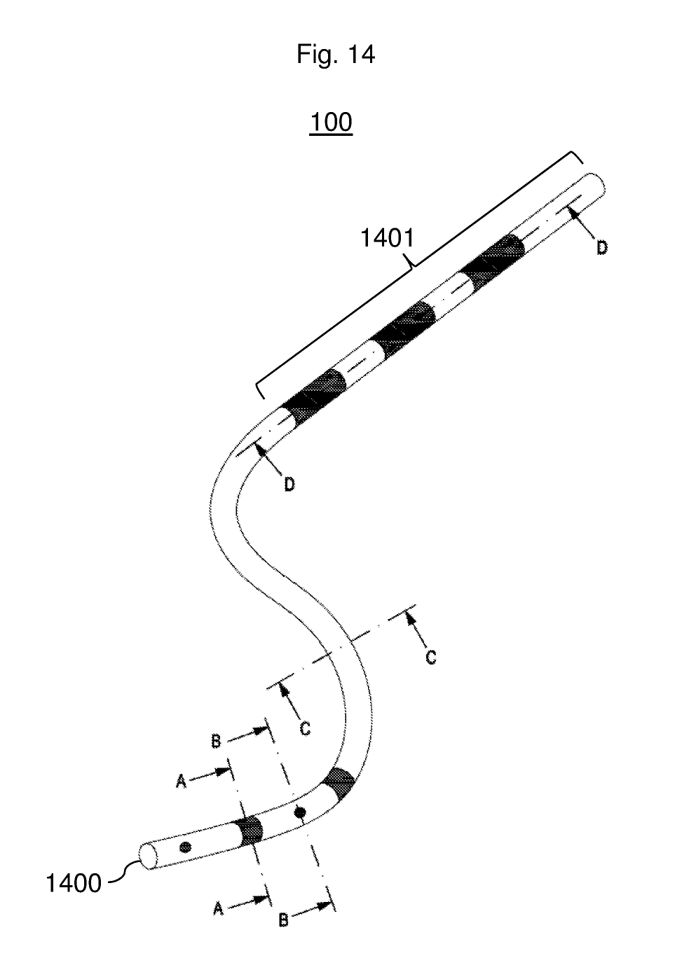

FIG. 14 a diagrammatic view of a lead with a composite according to the invention;

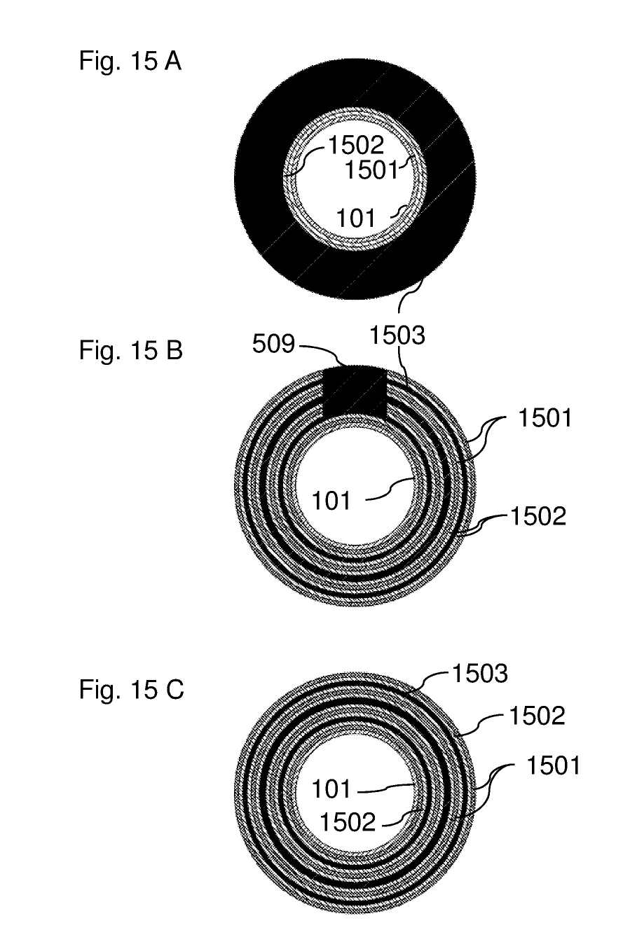

FIG. 15A a diagrammatic view of section A-A in FIG. 14;

FIG. 15B a diagrammatic view of section B-B in FIG. 14;

FIG. 15C a diagrammatic view of section C-C in FIG. 14; and

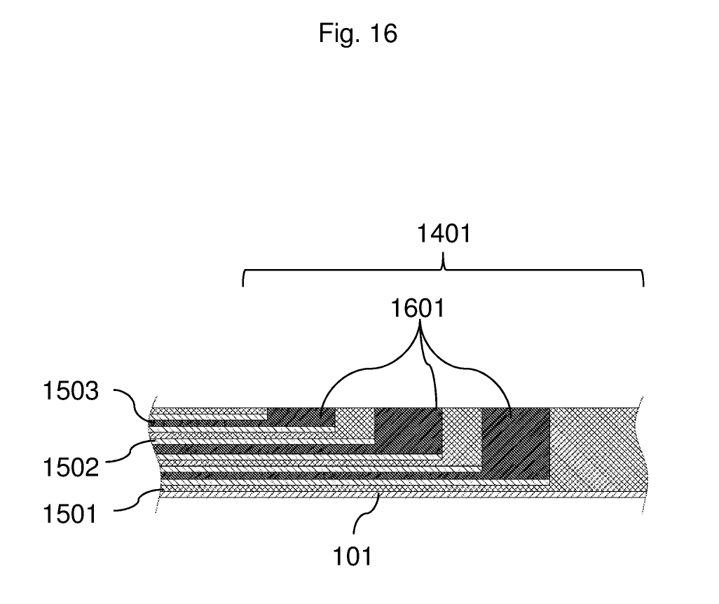

FIG. 16 a diagrammatic view of longitudinal section D-D in FIG. 14.

FIG. 1 shows a diagrammatic sectional view of a composite 100 according to the invention. The composite 100 contains a substrate 101 on which a first layer 102 is superimposed. The first layer 102 is composed of a polymer, here PMMA, and a plurality of electrically conductive particles, here a plurality of silver nanowires. Moreover, the first layer 102 contains a first layer surface 103, which is directly adjacent to the substrate 101. In a first region 104 of the first layer 102, the first layer 102 at a first distance 105 of 2 μm from the first layer surface 103 is characterized by a first content 403 of the electrically conductive particles of 2% based on a section 107 through the first layer 102 at the first distance 105. Moreover, in the first region 104, the first layer 102 at a further distance 106, which is equal to a thickness of the first layer 102 of 4 μm, is characterized by a further content of the electrically conductive particles of 50% based on a layer surface of the first layer 102, which is located at the further distance 106 from the substrate 101. Accordingly, the first content 403 is 48% less than the further content 404, and the first distance 105 is 2 μm less than the further distance 106. The substrate 101 is composed of poly(para-xylylene), also referred to a parylene N.

FIG. 2 shows a diagrammatic sectional view of a further composite 100 according to the invention. The composite 100 according to FIG. 2 is configured like the composite 100 according to FIG. 1, wherein the first distance 105 of the composite 100 according to FIG. 2 is 0 μm and thus lies on the first layer surface 103. The first content 403 of the electrically conductive particles is 0% based on the first layer surface 103. Furthermore, the first layer 102 of the composite 100 according to FIG. 2 at a second distance 201 of 2 μm is characterized by a second content 406 of the electrically conductive particles of 30% based on a section through the first layer 102 at the second distance 201.

FIG. 3 shows a diagrammatic sectional view of a further composite 100 according to the invention. The composite 100 according to FIG. 3 is configured like the composite 100 according to FIG. 2, wherein the polymer is PE and the electrically conductive particles are carbon nanotubes. In the first region 104 of the first layer 102, the first layer 102 at the first distance 105 of 0 μm from the first layer surface 103 is characterized by a first content 403 of the electrically conductive particles of 0% based on the first layer surface 103. Moreover, in the first region 104, the first layer 102 at the further distance 106, which is equal to a thickness of the first layer 102 of 12 μm, is characterized by a further content of the electrically conductive particles of 80% based on a further layer surface 405. Accordingly, the first content 403 is 80% less than the further content 404, and the first distance 105 is 12 μm less than the further distance 106. Furthermore, the first layer 102 of the composite 100 according to FIG. 3 at the second distance 201 of 2 μm is characterized by a second content 406 of the electrically conductive particles of 30% based on a section through the first layer 102 at the second distance 201. Moreover, the first layer 102 of the composite 100 according to FIG. 3 is characterized at a third distance of 6 μm by a third content of the electrically conductive particles of 40% based on a section through the first layer 102 at the third distance; is characterized at a fourth distance of 8 μm by a fourth content of the electrically conductive particles of 50% based on a section through the first layer 102 at the fourth distance; and is characterized at a fifth distance of 10 μm by a fifth content of the electrically conductive particles of 60% based on a section through the first layer 102 at the fifth distance. In the first region 104, a content 401 of the electrically conductive particles from the first distance 105, i.e. from the first layer surface 103, increases over the second distance 201, the third distance, the fourth distance, and the fifth distance until the further distance 106 is reached. Here, the further distance 106 is equal to a thickness of the first layer 102 of 12 μm and is thus on a further layer surface 405 opposite the first layer surface 103. The carbon nanotubes are characterized by a diameter of 12 nm and a length of 30 μm. A graph of a function of the content 401 of the electrically conductive particles in the first layer 102 from a distance 402 from the first layer surface 103 is shown in FIG. 4.

FIG. 4 shows a graphic representation of the content 401 of the electrically conductive particles in the first layer 102 of the composite 100 in FIG. 3 over the distance 402 from the first layer surface 103. FIG. 4 shows that the content 401 of the electrically conductive particles along a straight line from the first layer surface 103 to the further layer surface 405 is a monotonically increasing function of the distance 402 from the first layer surface 103. Here, the function has 5 steps.

FIG. 5 shows a diagrammatic sectional view of a further composite 100 according to the invention. The composite 100 according to FIG. 5 is configured like the composite 100 according to FIG. 3, wherein the composite 100 further contains an additional layer 501. The additional layer 501 is superimposed on the first layer 102 in a first partial area 507 of the first layer 102. Here, the additional layer 501 contains an additional first layer surface 502, which is adjacent to the further layer surface 405 of the first layer 102. The additional layer 501 is composed of the polymer, PE, and an additional plurality of the electrically conductive particles, the above-described carbon nanotubes. In a further region 506 of the additional layer 501, the additional layer 501 at an additional first distance 504 from the additional first layer surface 502 is characterized by an additional first content of the electrically conductive particles of 60% based on a section through the additional first layer 501 at the additional first distance 504. The additional first distance 504 is 2 μm. Moreover, the additional layer 501 in the further region 506 at an additional second distance of 4 μm from the additional first layer surface 502 is characterized by an additional second content of the electrically conductive particles of 50% based on a section through the additional first layer 501 at the additional second distance. Furthermore, the additional layer 501 in the further region 506 at an additional third distance of 6 μm from the additional first layer surface 502 is characterized by an additional third content of the electrically conductive particles of 10% based on a section through the additional first layer 501 at the additional third distance. In addition, the additional layer 501 in the further region 506 at an additional further distance 505 of 8 μm from the additional first layer surface 502 is characterized by an additional further content of the electrically conductive particles of 0% based on a section through the additional first layer 501 at the additional further distance 505. In this case, the additional further distance 505 lies on an additional further layer surface 503 of the additional layer 501. The additional further layer surface 503 lies opposite the additional first layer surface 502. The additional further distance 505 is thus equal to a thickness of the additional layer 501. The additional further layer surface is electrically deactivated. In a further partial area 508 adjacent to the first partial area 507 of the first layer 102, a contacting layer 509 is superimposed on the first layer 102 such that the contacting layer 509 is adjacent to the further layer surface 405. The contacting layer 509 is composed of the polymer and the carbon nanotubes, wherein the contacting layer 509 has a content of 90 vol.-% of the carbon nanotubes based on the total volume of the contacting layer 509. On a surface 510 of the contacting layer 509 facing away from the first layer 102, the contacting layer 509 is thus characterized by a lower contact resistance than the additional layer 501. The contacting layer 509 is composed of two areas separated from each other by the additional layer 501, both of which electrically contact the further layer surface 405 of the first layer 102 and thus constitute electrodes for electrical contacting.

FIG. 6 shows a diagrammatic sectional view of an apparatus 600 according to the invention with a graphical representation of a content 401 of electrically conductive particles. The apparatus 600 contains a substrate 101 and a coating 602. The substrate 101 contains a substrate surface 601 and is composed of silicone. The coating 602 contains a first surface 603 and a further surface 604 lying opposite the first surface 603. Here, the coating 602 is superimposed on the substrate 101 such that the first surface 603 is adjacent to the substrate surface 601. The further surface 604 accordingly faces away from the substrate 101. The coating 602 is composed of a polymer and a plurality of electrically conductive particles. The polymer is PEDOT. The electrically conductive particles are longitudinally extended gold wires with a diameter of 75 nm and a length of 15 μm. The coating 602 is characterized on the first surface 603 by a content 401 of the electrically conductive particles of 0% based on the first surface 603. Moreover, the coating contains a first partial volume 605, which is characterized by a content 401 of the electrically conductive particles of 80 vol.-% based on the volume of the coating 602 in the first partial volume 605. In a first region 606 of the coating 602, the coating 602 is characterized in that along a straight line 607 running from the first surface 603 to the further surface 604, a content 401 of the electrically conductive particles in the coating 602 is a function 609 of a position 608 on the straight line 607 with at least one first local maximum 610. For illustrative purposes, FIG. 6 shows a diagram with a graph of the function 609 at the right next to the representation of the apparatus. The first local maximum 610 is contained by the first partial volume 605 and is accordingly at 80 vol.-%. The function 609 decreases in 3 steps 612 from the first local maximum 610 to one global minimum 611 each adjacent in the direction of the first surface 603 and in the direction of the further surface 604 respectively. Moreover, the coating 602 on the further surface 604 is characterized by a content 401 of the electrically conductive particles of 0% based on the further surface 604. The first partial volume 605 is configured in sheetlike fashion and extends perpendicularly to the image plane of FIG. 6.

FIG. 7 shows a diagrammatic sectional view of a further apparatus 600 according to the invention. The apparatus 600 according to FIG. 7 is configured like the apparatus 600 of FIG. 6, wherein the apparatus 600 of FIG. 7 contains the first partial volume 605 and a further partial volume 701. In the first partial volume 605, the function 609 has the first local maximum 610 at 0 vol.-% based on the total volume of the first partial volume 605, and in the further partial volume 701, the function 609 has a further local maximum at 80 vol.-% based on the total volume of the further partial volume 701. Moreover, the total further partial volume 701 is characterized by a content 401 of the electrically conductive particles in a range of 80 vol.-% based on the volume of the coating 602 in the further partial volume 701. The function 609 decreases in 3 steps from the further local maximum to one minimum 611 each adjacent in the direction of the first surface 603 and in the direction of the further surface 604 respectively. A minimum 611 of the function 609 is thus located between the first partial volume 605 and the further partial volume 701. In this minimum 611, the content 401 of the electrically conductive particles is 0% based on a section through the coating 602. The further partial volume 701 is thus electrically insulated from the first partial volume 605. Analogously to the first partial volume 605, the further partial volume 701 is configured in sheetlike fashion and extends perpendicularly to the image plane of FIG. 7. The first partial volume 605 and the further partial volume 701 respectively form electrical conductors inside the coating 602. The coating 602 is therefore a two-phase electrical conductor.

FIG. 8 shows a flow chart of a method 800 according to the invention. The method 800 contains as a method step a) 801 the provision of a substrate 101 and 3 compositions. The substrate 101 in turn contains a substrate surface 601 Each of the 3 compositions contains a polymer, here PEDOT:PSS, and a plurality of electrically conductive particles, here silver flakes, in a particle content based on the weight of the respective composition. The 3 compositions are characterized in that the respective particle contents differ from one another. Composition 1 has a particle content of 0 vol.-% based on the total volume of composition 1. Composition 2 has a particle content of 30 vol.-% based on the total volume of composition 2. Composition 3 has a particle content of 60 vol.-% based on the total volume of composition 3. In a method step b) 802 downstream of the method step a) 801, the substrate surface 601 is first immersed in the composition 1 and thus wetted with a portion of composition 1. This portion of the composition 1 is then cured by heating to 100° C., thus obtaining a first layer that is superimposed on the substrate surface 601. After this, a surface of the first layer is immersed in the composition 2 and thus wetted with a portion of the composition 2. This portion of the composition 2 is then in turn cured by heating to 100° C., thus obtaining a second layer that is superimposed on the substrate surface 601 and the first layer. Furthermore, a surface of the second layer is immersed in the composition 3 and thus wetted with a portion of composition 3. This portion of the composition 3 is in turn cured by heating to 100° C., thus obtaining a third layer that is superimposed on the substrate surface 601, the first layer and the second layer. Moreover, a surface of the third layer is in turn immersed in the composition 3 and thus wetted with a portion of the composition 3. This portion of the composition 3 is then in turn cured by heating to 100° C., thus obtaining a fourth layer that is superimposed on the substrate surface 601, the first layer, the second layer and the third layer.

FIG. 9 shows a flow chart of a further method 800 according to the invention. The method 800 according to FIG. 9 contains the method steps a) 801 and b) 802 according to the method 800 of FIG. 8, as well as a method step c) 901. In the method step c) 901, a surface of the fourth layer is immersed in composition 2 and thus wetted with a further portion of composition 2. This portion of composition 2 is then cured by heating to 100° C., thus obtaining a further second layer. Furthermore, a surface of the further second layer is immersed in composition 1 and thus wetted with a further portion of the composition 1. This portion of the composition 1 is then cured by heating to 100° C., thus obtaining a further first layer.

FIG. 10 shows a flow chart of a further method 800 according to the invention. The method 800 according to FIG. 10 contains the method steps a) 801, b) 802 and c) 901 according to the method 800 of FIG. 9, as well as a method step d) 1001. In the method step d) 1001, a surface of the further first layer is electrically deactivated. This is carried out by partial halogenation of the surface.

FIG. 11 shows a diagrammatic view of a 3D printer 1100 according to the invention. The 3D printer 1100 is configured to produce the apparatus 600 according to FIG. 7. For this purpose, the 3D printer 1100 contains a nozzle 1101 with a nozzle opening 1102 having a diameter of 500 nm.

FIG. 12A shows a diagrammatic view of an electrical component 1200 according to the invention. The electrical component 1200 is a capacitor containing the apparatus 600 according to FIG. 7.

FIG. 12B shows a diagrammatic view of an electrical device 1201 according to the invention containing 3 electrical components 1200 according to the invention.

FIG. 13 shows a diagrammatic sectional view of a further apparatus 600 according to the invention. The apparatus 600 contains a substrate 101 and a coating 602. The substrate 101 contains a substrate surface 601 and is composed of polycarbonate. The coating 602 contains a first surface 603 and a further surface 604 opposite the first surface 603. Here, the coating 602 is superimposed on the substrate 101 such that the first surface 603 is adjacent to the substrate surface 601. The further surface 604 accordingly faces away from the substrate 101. The coating 602 is composed of a polymer and a plurality of electrically conductive particles. The polymer is SU-8. The electrically conductive particles are carbon nanotubes with a diameter of 12 nm and a length of 30 μm. The coating 602 is characterized on the first surface 603 by a content 401 of the electrically conductive particles of 0% based on the first surface 603. Moreover, the coating contains a first partial volume 605, which is characterized by a content 401 of the electrically conductive particles of 80 vol.-% based on the volume of the coating 602 in the first partial volume 605. In a first region 606 of the coating 602, the coating 602 is characterized in that along a straight line 607 that runs from the first surface 603 to the further surface 604, a content 401 of the electrically conductive particles in the coating 602 is a function 609 of a position 608 on the straight line 607 with at least one first local maximum 610. The first local maximum 610 is contained by the first partial volume 605 and accordingly is at 80 vol.-%. The function 609 decreases in 5 steps 612 from the first local maximum 610 to one global minimum 611 each adjacent in the direction of the first surface 603 and in the direction of the further surface 604 respectively. Here, the minima 611 are on the first surface 603 and the further surface 604 respectively. On the further surface 604, the coating 602 is characterized by a content 401 of electrically conductive particles of 0% based on the further surface. In FIG. 13, the coating 602 contains a further region 1301 into which the first partial volume 605 extends. In the further region 1301, the first partial volume 605 contains the further surface 604 of the coating 602. The first partial volume 605 can thus be electrically contacted on the further surface 604 in the further region 1301.

FIG. 14 shows a diagrammatic view of a lead 1400 with a composite 100 according to the invention. Such leads 1400 are used for example in implantable cardiac pacemakers as a flexible electrical connecting element between the pulse generator and the electrodes. In this case, the lead extends from the implantation site of the cardiac pacemaker, frequently under the collar bone, to the cardiac tissue to be stimulated. Such leads 1400 are multiphase electrical conductors that must be biocompatible, corrosion-resistant, flexible, mechanically strong, and show extremely good electrical conductivity. Here, the lead 1400 contains a substrate 101 of the composite 100. The substrate 101 is mechanically flexible and is composed of silicone. Moreover, sections A-A, B-B, C-C and D-D are depicted in the figure. Views of these sections are shown in FIGS. 15a) through c) and FIG. 16. One end of the lead 1400 is configured as a plug 1401. By means of this plug 1401, the lead 1400 can be connected to an analysis device, and measurement parameters such as pressure, temperature, current or a position of the lead 1400 can be measured and read out.

FIG. 15A shows a diagrammatic view of section A-A in FIG. 14. In this section, successive layers 1501, 1502, 1503 are superimposed in that order on the substrate 101 as follows: a layer 1501 with a content of electrically conductive particles of 0 vol.-% based on the total volume of the layer 1501; a layer 1502 with a content of electrically conductive particles of 50 vol.-% based on the total volume of the layer 1502; a layer 1503 with a content of electrically conductive particles of 80 vol.-% based on the total volume of the layer 1503. Each of the layers 1501, 1502 and 1503 consists of a polymer and the electrically conductive particles in the contents given above respectively. The layers 1501, 1502 and 1503 thus form a first layer 102 according to the composite 100 of the invention. For all of the layers 1501, 1502 and 1503 in FIGS. 14 through 16, the polymer is silicone and the electrically conductive particles are silver flakes.

FIG. 15B shows a diagrammatic view of section B-B in FIG. 14. In this section, successive layers 1501, 1502, 1503 are superimposed in that order on the substrate 101 as follows: a layer 1501 with a content of electrically conductive particles of 0 vol.-% based on the total volume of the layer 1501; a layer 1502 with a content of electrically conductive particles of 50 vol.-% based on the total volume of the layer 1502; a layer 1503 with a content of electrically conductive particles of 80 vol.-% based on the total volume of the layer 1503; a further layer 1502; a further layer 1501; a further layer 1502; a further layer 1503; a further layer 1502; a further layer 1501; a further layer 1502; a further layer 1503; a further layer 1502 and a further layer 1501. The layers 1501, 1502 and 1503 respectively consist of a polymer and the electrically conductive particles in the contents given above respectively. The layers 1501, 1502, 1503 closest to the substrate 101 thus form a first layer 102 according to the composite 100 of the invention. Moreover, the layers 1502 and 1501 following the first layer 102 form an additional layer 501 according to the composite 100 of the invention. In an upper area of the section of FIG. 15B, a further partial area 508 according to the invention can be seen, in which a contacting layer 509 is superimposed on the first layer 102. The contacting layer 509 is composed of the polymer and a content of 80 vol.-% of the electrically conductive particles. The electrically conductive layers 1503 can thus be electrically contacted from outside of the lead 1400 via the contacting layer 509. Moreover, the layers 1501, 1502 and 1503 in FIG. 15B form an apparatus 600 according to the invention. Here, all of the layers 1501, 1502, 1503 form a coating 602. The innermost layer 1503 forms a first partial volume 605, and the more external layers 1503 respectively form a further partial volume 701.

FIG. 15C shows a diagrammatic view of section C-C in FIG. 14. In this section, successive layers 1501, 1502, 1503 are superimposed in that order on the substrate 101 as follows: a layer 1501 with a content of electrically conductive particles of 0 vol.-% based on the total volume of the layer 1501; a layer 1502 with a content of electrically conductive particles of 50 vol.-% based on the total volume of the layer 1502; a layer 1503 with a content of electrically conductive particles of 80 vol.-% based on the total volume of the layer 1503; a further layer 1502; a further layer 1501; a further layer 1502; a further layer 1503; a further layer 1502; a further layer 1501; a further layer 1502; a further layer 1503; a further layer 1502 and a further layer 1501. The layers 1501, 1502 and 1503 respectively consist of a polymer and the electrically conductive particles in the contents given above respectively. The layers 1501, 1502, 1503 closest to the substrate 101 thus form a first layer 102 according to the composite 100 of the invention. Moreover, the layers 1502 and 1501 following the first layer 102 form an additional layer 501 according to the composite 100 of the invention. Moreover, the layers 1501, 1502 and 1503 in FIG. 15C form an apparatus 600 according to the invention. Here, all of the layers 1501, 1502, 1503 form a coating 602. The innermost layer 1503 forms a first partial volume 605, and the more external layers 1503 respectively form a further partial volume 701.

FIG. 16 shows a diagrammatic view of longitudinal section C-C in FIG. 14. The figures shows a longitudinal section through the plug 1401 of the lead 1400. The layers 1501, 1502, 1503 are also shown. These are the same layers shown in FIG. 15C. Moreover, each of the layers 1503 is electrically contactable via electrical contacts 1601 of the plug 1401.

### LIST OF REFERENCE SIGNS

100 Composite according to the invention

101 Substrate

102 First layer

103 First layer surface

104 First region

105 First distance

106 Further distance

107 Section

201 Second distance

401 Content of electrically conductive particles

402 Distance from the first layer surface

403 First content of electrically conductive particles

404 Further content of electrically conductive particles

405 Further layer surface

406 Second content of electrically conductive particles

501 Additional layer

502 Additional first layer surface

503 Additional further layer surface

504 Additional first distance

505 Additional further distance

506 Further region

507 First partial area

508 Further partial area

509 Contacting layer

510 Surface of the contacting layer

600 Apparatus according to the invention

601 Substrate surface

602 Coating

603 First surface

604 Further surface

605 First partial volume

606 First region of the coating

607 Straight line

608 Position on the straight line

609 Function

610 First local maximum

611 Minimum

612 Step of the function

701 Further partial volume

800 Method according to the invention

801 Method step a)

802 Method step b)

901 Method step c)

1001 Method step d)

1100 3D printer according to the invention

1101 Nozzle

1102 Nozzle opening

1200 Electrical component according to the invention

1201 Electrical device according to the invention

1301 Further region of the coating

1400 Lead

1401 Plug

1501 Layer with 0 vol.-% electrically conductive particles

1502 Layer with 40 vol.-% electrically conductive particles

1503 Layer with 80 vol.-% electrically conductive particles

1601 Electrical contacts

1. A composite comprising as mutually superimposed layers of a series of layers
a) a substrate, and
b) a first layer;
wherein the first layer comprises
i) a first layer surface,
ii) a polymer, and
iii) a plurality of electrically conductive particles;
wherein the first layer surface is adjacent to the substrate;
wherein at least in a first region, the first layer at a first distance from the first layer surface is characterized by a first content of the electrically conductive particles;
wherein at least in the first region, the first layer at a further distance from the first layer surface is characterized by a further content of the electrically conductive particles;
wherein the first content is less than the further content; and
wherein the first distance is less than the further distance.

2. The composite according to claim 1, wherein at least in the first region, the first layer at a second distance from the first layer surface is characterized by a second content of the electrically conductive particles;
wherein the second content is less than the further content and more than the first content; and
wherein the second distance is less than the further distance and more than the first distance.

3. The composite according to claim 1, wherein the first layer in the first region is characterized in that a content of the electrically conductive particles increases from the first distance to the further distance.

4. The composite according to claim 1, wherein the first layer further comprises a further layer surface opposite the first layer surface,
wherein the first layer in the first region is characterized in that a content of the electrically conductive particles along a straight line from the first layer surface to the further layer surface is a monotonically increasing function of a distance from the first layer surface.

5. The composite according to claim 1, wherein the first layer on the first layer surface is characterized by a content of the electrically conductive particles in a range of 0 to 5% based on the first layer surface.

6. The composite according to claim 1, wherein the electrically conductive particles comprise a substance selected from the group of gold, silver, palladium, platinum, carbon, and a combination of at least two thereof.

7. The composite according to claim 1, wherein the polymer is selected from the group composed of silicone, an electrically conductive polymer, a lacquer, a polyaromatic, a thermoplastic, a resin, and a combination of at least two thereof.

8. The composite according to claim 1, wherein the substrate comprises a substance selected from the group of a plastic, a plastic mixture, and a metal, and a combination of at least two thereof.

9. The composite according to claim 1, wherein the substrate is contained by one selected from the group composed of a medical device, a medical aid, an electrical device, and a combination of at least two thereof.

10. The composite according to claim 1, wherein the substrate is selected from the group composed of a tube, a catheter, a wire, a needle, a probe, an implant, a film, a cannula, a lead, and a combination of at least two thereof.

11. The composite according to claim 1, wherein the composite is selected from the group composed of a medical device, a medical aid, a plug, a socket, and a combination of at least two thereof.

12. An apparatus containing a substrate and a coating, wherein the substrate contains a substrate surface;
wherein the coating
a) comprises a first surface and a further surface,
wherein the first surface is adjacent to the substrate surface, and
wherein the further surface faces away from the substrate;
b) comprises a polymer and a plurality of electrically conductive particles;
c) is characterized on the first surface by a content of the electrically conductive particles in a range of 0 to 20% based on the first surface;
d) comprises a first partial volume; and
e) is characterized in the first partial volume by a content of the electrically conductive particles in a range of 1 to 100 vol.-% based on the volume of the coating in the first partial volume;
wherein in a first region of the coating,
i) the coating is characterized in that along a straight line from the first surface to the further surface, a content of the electrically conductive particles in the coating is a function of a position on the straight line with at least one first local maximum,
wherein the first local maximum is contained by the first partial volume, and
wherein the function decreases continuously or in at least 2 steps from the first local maximum to one adjacent minimum each in the direction of the first surface and in the direction of the further surface; and
ii) the coating on the further surface is characterized by a content of the electrically conductive particles in a range of 0 to 20% based on the further surface.

13. A method comprising as method steps
a) providing a substrate and n compositions;
wherein the substrate contains a substrate surface,
wherein each of the n compositions contains a polymer and a plurality of electrically conductive particles in a particle content based on the weight of the respective composition, and
wherein the n compositions are characterized in that the respective particle contents of the n compositions differ from one another; and
b) superimposing on the substrate surface of at least one first portion each of the n compositions,
wherein the superimposing of least the first portions of the n compositions takes place successively,
wherein after each superimposing of at least the first portion of one of the n compositions, the at least one first portion is cured,
wherein at least the first portions of the n compositions are superimposed in a series of increasing particle contents,
wherein a first series of layers is obtained containing from the substrate surface in a layer sequence direction a first to an n^{th} layer of mutually superimposed layers, and
wherein n is a natural number greater than 1.

14. An apparatus obtained by the method according to claim 13.

15. An electrical component comprising a composite according to claim 1.

16. An electrical device comprising a composite according to claim 1.

17. A 3D printer configured to produce a composite according to claim 1.

18. The use of a composition comprising a polymer and a plurality of electrically conductive particles, for electrical contacting of a coating superimposed on a substrate.

19. An electrical component comprising an apparatus according to claim 12.

20. An electrical device comprising an apparatus according to claim 12.

21. An electrical device comprising an electrical component according to claim 15.

22. A 3D printer configured to produce an apparatus according to claim 12.
